(19) United States
(12) Patent Application Publication
(10) Pub. No.: US 2020/0221951 A1
(43) Pub. Date: Jul. 16, 2020

(54) METHODS AND SYSTEMS FOR AN INTEGRATED TELEHEALTH PLATFORM

(71) Applicants: Ravi Amble, San Jose, CA (US); Laurence Edward England, Morgan Hill, CA (US); Radhika Padmanabhan, Saratoga, CA (US); G. Meenakshi Sundaram, Chennai (IN)

(72) Inventors: Ravi Amble, San Jose, CA (US); Laurence Edward England, Morgan Hill, CA (US); Radhika Padmanabhan, Saratoga, CA (US); G. Meenakshi Sundaram, Chennai (IN)

(21) Appl. No.: 16/672,496

(22) Filed: Nov. 3, 2019

Related U.S. Application Data
(60) Provisional application No. 62/755,317, filed on Nov. 2, 2018.

(51) Int. Cl. A61B 5/00; G16H 10/60; G16H 40/67; G16H 80/00; G16H 50/20; G16H 30/40; G16H 30/20; G06N 20/00; G06K 9/00; G06K 9/46

(52) U.S. Cl. CPC A61B 5/0022; G16H 10/60; G16H 40/67; G16H 80/00; G16H 50/20; G06K 2009/4695; G16H 30/20; G06N 20/00; G06K 9/00711; G06K 9/46; G16H 30/40

(57) ABSTRACT

In one aspect, a computerized method useful for implementing a language neutral virtual assistant includes the step of providing a set of digital medical devices. The set of digital medical devices obtains a specified medical sensor information from a patient and communicates the specified medical sensor information to one or more remote entities. The method includes the step of providing a set of tele-health devices. The set of tele-health devices communicates an audio-video data of a tele-health session to the one or more remote entities. The method includes the step of creating an open Device Integration Layer (DIL), wherein the DIL communicates with the set of digital medical devices. The DIL communicates with the set of tele-health devices in a tele-health session. The method includes the step of with the DIL, parsing and translating a digital output of the medical device data of the set of digital medical devices and the set of tele-health devices. The method includes the step of with the DIL, optimizing and compressing the translated digital output of the medical device data of the set of digital medical devices and the set of tele-health devices. The method includes the step of with the DIL, integrating the translated digital output of the set of digital medical devices regardless of any output format of any of the digital medical devices. The method includes the step of providing a user interface of a medical provider's computing system. The integrated data of the set of digital medical devices is represent by each type of digital medical devices in a canonical form with the audio-video data of the tele-health session.

### CLAIM OF PRIORITY

This application claims priority to U.S. Provisional Application No. 62/755,317, titled INTEGRATED TELEHEALTH PLATFORM, and filed on 2 Nov. 2018. This application is incorporated herein by reference.

### BACKGROUND

Telehealth is the distribution of health-related services and information via electronic information and telecommunication technologies. It allows long-distance patient and clinician contact, care, advice, reminders, education, intervention, monitoring, and remote admissions. Telemedicine can involve the use of many different data sources (e.g. various medical sensors, user medical records, video streams, etc.). Telemedicine data can be subject to strict regulation by various government laws and agencies across a variety of jurisdiction. Furthermore, there is a need to format and present the telemedicine data in a manner that is useable by a medical service provide. Finally, telemedicine data can be acted upon immediately for real-time and near real time analysis, and/or be saved for later use for big-data type analysis in order to improve future telemedicine practices.

### BRIEF SUMMARY OF THE INVENTION

In one aspect, a computerized method useful for implementing a language neutral virtual assistant includes the step of providing a set of digital medical devices. The set of digital medical devices obtains a specified medical sensor information from a patient and communicates the specified medical sensor information to one or more remote entities. The method includes the step of providing a set of tele-health devices. The set of tele-health devices communicates an audio-video data of a tele-health session to the one or more remote entities. The method includes the step of creating an open Device Integration Layer (DIL), wherein the DIL communicates with the set of digital medical devices. The DIL communicates with the set of tele-health devices in a tele-health session. The method includes the step of with the DIL, parsing and translating a digital output of the medical device data of the set of digital medical devices and the set of tele-health devices. The method includes the step of with the DIL, optimizing and compressing the translated digital output of the medical device data of the set of digital medical devices and the set of tele-health devices. The method includes the step of with the DIL, integrating the translated digital output of the set of digital medical devices regardless of any output format of any of the digital medical devices. The method includes the step of providing a user interface of a medical provider's computing system. The integrated data of the set of digital medical devices is represent by each type of digital medical devices in a canonical form with the audio-video data of the tele-health session.

### BRIEF DESCRIPTION OF THE DRAWINGS

FIG. 1 illustrates an example process for implementing an integrated telehealth platform, according to some embodiments.

FIG. 2 illustrates an example device integration and acquisition system, according to some embodiments.

FIG. 3 illustrates an example tele-health platform, according to some embodiments.

FIG. 4 illustrates an example process for implementing a language neutral virtual assistant, according to some embodiments.

FIG. 5 depicts an exemplary computing system that can be configured to perform any one of the processes provided herein.

FIG. 6 is a block diagram of a sample computing environment that can be utilized to implement various embodiments.

FIG. 7 illustrates an example an integrated telehealth platform system, according to some embodiments.

The Figures described above are a representative set and are not an exhaustive with respect to embodying the invention.

### DESCRIPTION

Disclosed are a system, method, and article of manufacture for an integrated telehealth platform. The following description is presented to enable a person of ordinary skill in the art to make and use the various embodiments. Descriptions of specific devices, techniques, and applications are provided only as examples. Various modifications to the examples described herein can be readily apparent to those of ordinary skill in the art, and the general principles defined herein may be applied to other examples and applications without departing from the spirit and scope of the various embodiments.

Reference throughout this specification to ‘one embodiment,’ ‘an embodiment,’ ‘one example,’ or similar language means that a particular feature, structure, or characteristic described in connection with the embodiment is included in at least one embodiment of the present invention. Thus, appearances of the phrases ‘in one embodiment,’ ‘in an embodiment,’ and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### Definitions

Example definitions for some embodiments are now provided.

Application programming interface (API) can specify how software components of various systems interact with each other.

Deep learning is a family of machine learning methods based on learning data representations. Learning can be supervised, semi-supervised or unsupervised. Deep learning typically involves multi-layer neural networks, but may also embody other analytical methods such as Bayesian statistics, etc. Machine learning can also be employed.

Digital Imaging and Communications in Medicine (DICOM) is the standard for the communication and management of medical imaging information and related data. DICOM can be used for storing and transmitting medical images enabling the integration of medical imaging devices such as scanners, servers, workstations, printers, network hardware, and picture archiving and communication systems (PACS) from multiple manufacturers. Digital Imaging and Communications in Medicine (DICOM) is a standard for handling, storing, printing, and transmitting information in radiological images and other medical information between computers. It includes a file format definition and a network communications protocol. The communication protocol is an application protocol that uses TCP/IP to communicate between systems (DICOM workstation and PACS servers or PACS servers to rendering workstations). DICOM files can be exchanged between two entities that are capable of receiving image and patient data in DICOM format. The intention of DICOM is to define the communication capabilities to allow products supplied by different vendors to be interoperable and form an open, integrated diagnostic, treatment protocol. DICOM has been widely adopted by hospitals and is making inroads in smaller applications like dentists' and doctors' offices.

Electronic health record (EHR) is the systematized collection of patient and population electronically-stored health information in a digital format. These records can be shared across different health care settings. Records can be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information. EHR (sometimes known as EMR—Electronic medical record) can vary in exact structure and content across different vendors. This implementation normalizes the information to accommodate the variances.

Machine learning is a type of artificial intelligence (AI) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity and metric learning, and/or sparse dictionary learning.

Medical device connectivity is the establishment and maintenance of a connection through which data is transferred between a medical device, such as a patient monitor, and an information system.

Example Methods

An example method for creating a complete telehealth delivery ecosystem by bringing together and integrating several areas necessary for a holistic remote health care platform is provided. The method can provide an integrated ecosystem comprising of the following systems: humans (e.g. patients, providers, clinicians, family, specialty physicians and others); medical and clinical tools and services (e.g. medical devices support for relevant diagnosis for the demography, EHR and systems, audio-video sessions for communication); organizations (e.g. facilities, hospitals, third party healthcare providers, etc.); technologies (e.g. networking, video conferencing, telemetry support at diagnostic quality information delivery, secure cloud infrastructure for medicine; diagnostic level remote viewing abilities; and critically, clinical and systemic augmented intelligence—for physicians, clinicians, administrators and IT specialists); etc.

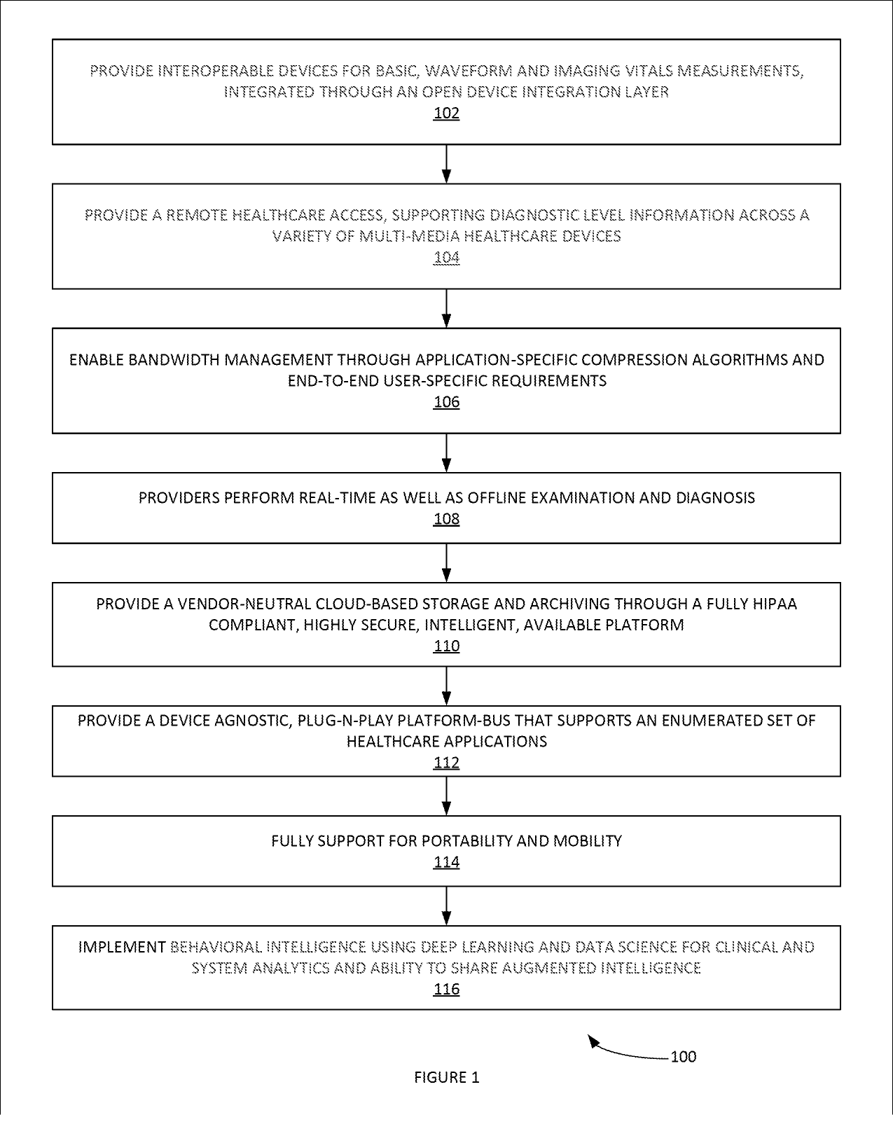

FIG. 1 illustrates an example process 100 for implementing an integrated telehealth platform, according to some embodiments. Process 100 can provide a standardized approach to build and maintain an integrated telehealth platform where all the components and parts of a healthcare delivery system can work in tandem in a seamless, user friendly manner for the participants regardless of geographical locations. Process 100 can provide the various processes and open interfaces through which other third parties can integrate into this common, standard solution.

In step 102, process 100 can provide interoperable devices for basic, waveform and imaging vital measurements, integrated through an open device integration layer. Step 102 can enable multiple communication channels for transmitting device telemetry data along with audio-video data for real-time consultation and diagnosis. Step 102 can enable the use of existing devices in the market by creating an open Device Integration Layer (DIL) (e.g. see infra). The DIL can allow for the integration of any third-party medical (digital) device regardless of the output format to integrate with DIL and communicate with remote entities in a tele-health session. Step 102 can be parse and translate digital output of various types of medical device data. Accordingly, step 102 can provide an interface to represent each type of device in a canonical form. Upon translation, the device data can be optimized, compressed and transported. In a similar fashion, audio/video channel for consultation along with the additional features, such as file transfer, screen sharing, recording with permissions, etc. can be optimized, compressed and transported. A transportation engine can be provided. The transportation engine can transmit the data in the correct method/frequency/security to the cloud servers for distribution to remote entities.

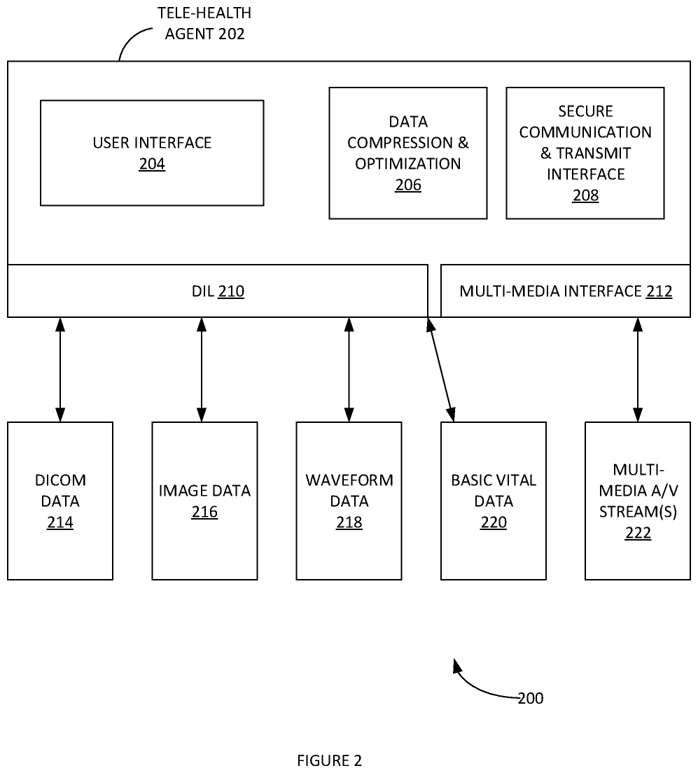

FIG. 2 illustrates an example device integration and acquisition system 200, according to some embodiments. Device integration and acquisition system 200 can include a telehealth agent 202. telehealth agent 202 is the module present at the patient clinic. Telehealth agent 202 acquires the required tele-health information. Telehealth agent 202 procures various device data, converts them into canonical form, enables the compression and transmission based on the nature and priority of various data types. In addition, telehealth agent 202 also handles other forms of tele-health data procurement and transmission, such as audio-video conference streams and required feature sets. System 200 can define a message structure for each category of device based on device-type, etc. Telehealth agent 202 functionalities pertain to device handling and can include, inter alia: live tele-medicine session between the patient clinic and physicians; obtaining device information from various configured and activated devices; identifying devices into categories based on audio/video streaming or data transmission; compressing and transmitting based on requirements; providing a separate connection for streaming for live video conference; providing a separate connection streaming for multiple imaging devices for video streaming of images received from frame-grabber-like device; providing a separate connection for transmission for data-type devices. Telehealth agent 202 can include data compression and optimization 206 secure communication and transmit interface 208.

Device Integration Layer (DIL) 210 resides in the agent and is the interface with which other third-party devices integrate. It is noted that device manufacturers can create a device specific driver that can get instantiated within telehealth agent 202. Devices can be classified into different categories based on the output format of medical devices: basic vitals devices, waveform devices, imaging devices, streaming media devices, etc. Basic vital devices (e.g. blood pressures, pulse data, temperature data, O2 (blood oxygen level), weight, etc.) can send discrete data as input to the DIL. Waveform devices (e.g. EKG, ECG, etc.) can send data patterns as input. Imaging devices (e.g. radiology data, ultrasound data, digital X-ray data, otoscope, etc.) could can involve discrete images or stream of images. Streaming devices (e.g. digital camera systems, digital microphone systems, etc.) send audio and/or video streams as input. In this way, telehealth agent 202 can obtain DICOM data 214, image data 216, waveform data 218, basic vital data 220, etc. A multi-media interface 212 can be used by telehealth agent 202 to obtain multimedia a/v stream(s) 222, etc. The data 214-222 can be viewed via user interface 204.

Example device characteristics are now discussed. Basic-vitals devices can provide digital data through APIs. Basic vitals can be retrieved through telehealth agent 202. Data can be obtained via device drivers communicating with the DIL directly. The device can be interoperable with any form factor such as BT, Wi-Fi or other technologies.

Waveform devices should be able to provide digital data through APIs. Waveform device data output can be retrieved through telehealth agent 202. Waveform devices can use patterns of data stream based on device output protocol. Data can be obtained via device drivers communicating with DIL directly. Waveform devices can work with any form factor such as BT, Wi-Fi and/or other technologies.

Imaging devices can have two methods of sending information in telehealth agent 202. Live streaming can be utilized as well. Post-session image streaming can include post-session DICOM information. Streaming can occur through video grabbers where the device's output can be captured via display and streamed to a proprietary driver at telehealth agent 202. The video port can be connected to telehealth agent 202 and can handle the video streaming. Offline imaging data (DICOM) data 214 can be uploaded post session completion to telehealth agent 202 multi-media devices. Multi-media devices can include devices that stream audio, video or combination of both as output streams (e.g. multimedia a/v stream(s) 222, etc.). The handling of streaming media devices is similar in concept to standard audio/video conference; and differ in the handling of respective streams. Each stream, based on the device type and characteristics, can use different handling such as, inter alia: compression, rate of transmission, error correction, etc.

A live telemedicine session can be implemented. The live audio-video session between physicians, patients, and/or specialists, family members can be handled via telehealth agent 202 as well.

In one example, system 200 of telemetry devices connected to tele-clinic, according to some embodiments. System 200 can include a telemetry pipe for medical device data transmission. Within this pipe, each device can be encoded based on the nature of data output (e.g. waveform, images, digital media, etc.), clinical properties of device output required for diagnosis (ex. ECG, Ultrasound, MRI, X-ray, etc.). This can enable a configurable telemetry session for diagnostic quality transmission. A primary care teleclinic may comprise of devices (e.g. Temp, SPO2, BP, Pulse, ECG, Stethoscope, etc.), as well as, various audio-video consulting channels. The readings from a single teleclinic can be transmitted as a composite channel to the server. The properties of each of these channels can vary depending on the type of device, its characteristics and required clinical properties. System 200 can include a thin driver for each category of device and encode each device in its own channel based on requirements for that device and build a composite stream of data for telemetry transmission. This can enable medical device telemetry to cater to the specific needs of the medical and clinical community, thereby making quality diagnosis across telehealth systems seamless. System 200 can enable any teleclinic being used for patient treatment and diagnosis by a remote physician, the telemetry data transmission can provide diagnostic quality of data.

Returning to process 100, in step 104, process 100 can provide a remote healthcare access, supporting diagnostic level information across a variety of multi-media healthcare devices.

In step 106, process 100 can enable bandwidth management through application-specific compression algorithms and end-to-end user-specific requirements. Step 106 can detect bandwidth fluctuations, assess its impact on medical device data transmission, and recalibrate tele-health configurations is key to successful tele-heath delivery systems. Step 106 can provide dynamic bandwidth configuration, detection and management in tele-health networks. Step 106 can provide real-time ability to detect bandwidth variations. Step 106 can provide bandwidth requirements for various types of medical devices for telemetry communications. Step 106 can implement algorithms for auto-configuring the data transmission based on types of devices and nature of tele-health session. The principles developed are applicable for both input to servers from patient centers and distribution from the servers to user (ex. Physician) portals.

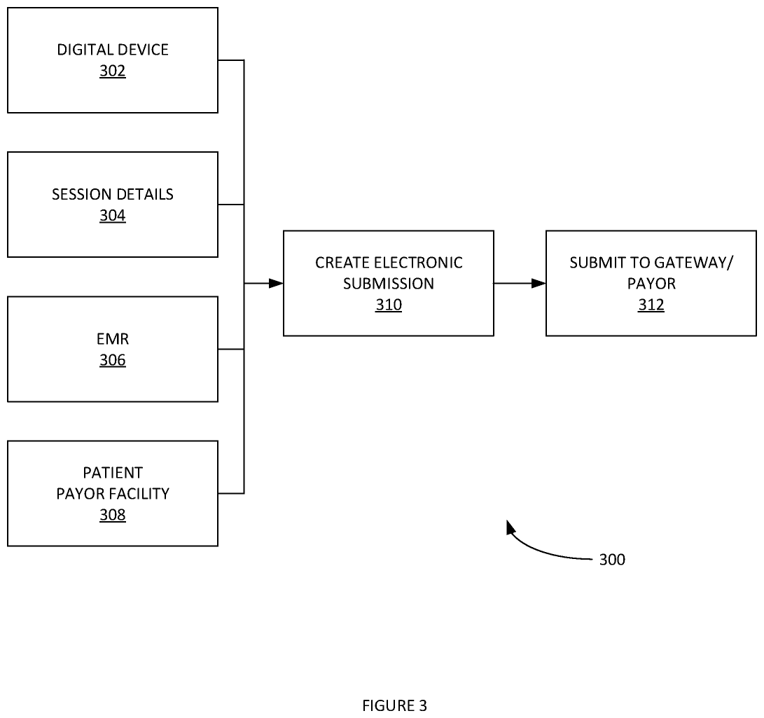

FIG. 3 illustrates an example tele-health platform 300, according to some embodiments. It is noted that tele-health networks can include a number of medical (e.g. digital) devices 302 that are required for complete screening of patients and an audio-video conference session details 304 for simultaneous consultation. Medical devices 302 can use low bandwidth and transmit data in discrete fashion. Platform 300 can provide a tele-health network that includes various types of devices, each with their own data transmission requirements to ensure diagnostic quality. This is accompanied with a live audio-video conference session for live consultation. This enables process 100 to evaluate the required bandwidth for any given session with specified (e.g. digital) medical devices. Along with this setup, when the nature of tele-health session is defined, process 100 can include algorithms to dynamically manage the priority of the devices for any given session. Platform 300's methods can be applicable in the input, as well as the distribution segments of process 100. When bandwidth fluctuates, platform 300 can enable the critical devices to continue transmitting data, while it stores the data form other devices in local cache. The local cache is synced to the servers when bandwidth becomes available for future review. When bandwidth falls below certain threshold that makes a critical device drop the quality below diagnostic levels, platform 300 can turn off the device's data transmission and begin maintaining the information in local cache for future sync to the servers. In this way, platform 300 can maintain the integrity of the data provided to the physicians at all times. In a similar manner, when rendering the device data to the remote physicians, platform 300 can the bandwidth and when able to transmit the data from the servers (e.g. in a cloud-computing environment) with diagnostic quality. Platform 300 can enable the transmission of any device data to physician. When multiple windows are concurrently streaming data from patient end to the physician, if bandwidth fluctuates, platform 300 can use the same priority algorithm in the distribution segment to ensure diagnostic quality within available bandwidth. Platform 300 can be used in tele-health session to manage required bandwidth at the tele-clinic housing patients and clinicians. In this way, platform 300 can manage the bandwidth and viewing modalities for diagnostic quality for remote physicians, specialty doctors, etc.

The system defines a role-based access schema for tele-health delivery that factors in pre-defined sets of views for any given role. The system can manage the views based on configuration, and available system and network resources for effective rendering. For example, when a physician conducts a tele-health session with remote patient in real-time and requires access to a bunch of telemetry information in addition to health records, audio-video session for viewing and consulting with a patient and others simultaneously, the algorithm allows for viewing of all the above data in multiple widgets in a portal screen based on the access rights of the physician with the patient and facility information. While the data is being transmitted in real-time, system and network resources are monitored, and the displayed widgets are scaled based on configuration, and priority. The algorithms for priority configuration can be developed based on analytics using information such as, inter alia: the nature of session; configured and activated devices; and/or a physician's preference over time. When a user accesses the session via the portal, available views can be presented based on the role and the access.

A framework for rendering medical devices can be provided. The framework can be utilized for the representation of widgets. Based on pre-determined priority views for the session, available bandwidth for communication and streaming, the power of the viewing entity, the in-session selections made by the user, the screen layout can be determined and presented to the user. It is noted that a live tele-health session view of multiple devices and connected data can be provided by process 100.

It is noted that process 100 can populate the user's screen with pre-configured widgets for the session. For example, a live conference feed can be seen as the initial widget. There can be active and available widgets presented at any time and the physician can select required widgets for viewing from among available widgets. Available widgets are those requested by the physician for which data is available. Active widgets are those that are being displayed in the portal. An available widget cannot consume bandwidth. Active widgets can use bandwidth based on the nature of data received from the servers. Information propagation and rendering at remote end. As the session progresses, a physician may use different types of live data—telemetry data from various medical devices from the patient end; audio-video channels form patient end as well as other participants in the session. When the clinician enables a device reading at the patient end, device data collection begins in a cloud-computing environment. Clinical data that has not yet been configured from the patient end does not appear in the widget schema. The data collection service engine in the cloud keeps polling for clinical data streams and can notify the corresponding session/user (e.g. the physician, etc.) when it finds data pertaining to any device available. The notification is received by the physician that a new widget is now ‘available’. The physician can make the widget ‘active’ by selecting the device.

When the bandwidth fluctuates and is unable to cater to the number of widgets being streamed, there can be an alert and the widget views can be automatically scaled based on viewing priorities to display the high priority views that can be accommodated within available bandwidth. In case of increase in bandwidth, additional widgets can be added automatically from available ones. It is noted that these methods can be used in tele-health viewing modalities for role-based access. The viewing modalities may differ based on the form factor (ex. smart phone, tablet, laptop, etc.) used for viewing by the portal users of the distribution engine. In one example, a physician may interview/examine a patient, the sub-specialty doctor, the family member, as well as several devices live telemetry information all at the same time.

Returning to process 100, in step 108, process 100 can enable providers to perform real-time as well as offline examination and diagnosis.

In step 110, process 100 can provide a vendor-neutral cloud-based storage and archiving through a fully HIPPA-compliant, highly secure, intelligent, available platform.

In step 112, process 100 can provide a device agnostic, plug-n-play platform-bus that supports an enumerated set of healthcare applications. Step 112 can provide an open workflow interface. The workflow interface can enable integration with other EHRs and workflow vendors.

It is noted that data from aspects of the system and participants can be collected and used to establish a cache of data to be interrogated. Patient data is collected digitally at every encounter of the system for participants within the system and is available for analytics. This presents an ever-increasingly complex set of analytics. There can be different levels of ‘analytics’ that are explored. At some of the higher analytical levels, there is an iterative approach that begins with a premise, then ask if the data necessary to support the premise, finally, looking at the collected data (e.g. filling in missing data as they are discovered) to test the premise. Along the way, one may observe unanticipated, unexpected discoveries. Hence, analytics can be an ongoing activity. Analytics can be used to produce actionable insights resulting in smarter decisions and better outcomes.

Analytics can be provided in four levels, each level builds upon a lower level. Descriptive Analytics can describe what happened. They can deliver statistics in a visually descriptive, insightful manner. Diagnostic analytics can describe why an event happen. This can identify the causes of an event. Predictive analytics can be used to predict future events. This can use historical information, establish predictions (e.g. within a confidence level) of what will happen. Prescriptive analytics can use historical information and corresponding results and outcomes, to provide courses action to produce desired outcomes. In this way, process 100 can present a set of actions that can produce a desired outcome (e.g. within a confidence level). Samples of captured information include, inter alia: session details; duration of session; number of participants; N-way (e.g. as it fluctuates over time facilities/organizations; information by site location/facility (Long term care organization, remote facility, etc.); information by provider's organization; captured digital device information (e.g. ECG/EKG; NIBP; respiratory rate; heart rate; oxygen saturation; body temperature; blood glucose; image analytics; etc.); etc. In a graduated approach, methods can generate statistics for each area and present to the actors of the system for all aspects. The methods and system of analytics can be used to yield insights into activities as well. With the information captured and managed digitally across multiple disciplines, cross domain analytics can be determined.

In step 114, process 100 can fully support for portability and mobility. For example, process 100 can use a hardware device that is small, concise required for portability/mobility.

In step 116, process 100 can implement behavioral intelligence using deep learning and data science for clinical and system analytics and ability to share augmented intelligence.

A discussion of how participants in a tele-health multi-way session is implemented in now provided. Capturing the perceptions of all participants within a telehealth session is obtained in two different modes—explicit and implicit. For the explicit mode, during a telemedicine session, participants can be presented with a small input area for comments or a simple ‘like’. The participants can anonymously direct their comments about active participants within the session and about the system itself. For example, in the telehealth session involving only a provider and a patient, the provider can establish comments and/or ‘likes’ towards the patient and the system. In a similar manner, the patient can establish anonymous comments and/or ‘likes’ towards the provider and about the system. These comments and likes can be saved in a database and tallied. Sentiment analysis can be used to process the written comments. Natural language processing, text analytics, and computational linguistics can be employed to identify and potentially quantify the affective sentiment states and subjective information. Information directed towards a given system participant can be gathered over time. Patterns can emerge and be identified. This can embody both subjective and objective information about the patient and the participants, as well as the system itself.

For the implicit mode, during a telemedicine session, two approaches can be employed—perform sentiment analysis on the dialog between the system actors and capture the navigation movements on the browser screen to determine the usage patterns of the system. In the implicit mode, the participants voices are recorded and saved digitally. Speech-to-text conversions can be performed, and the result can be used in sentiment analysis. Analytics performed on the collected data can be used as quality metrics (MACRA—Medicare Access and Chip Reauthorization Act of 2015 and MIPS—Merit-based Incentive Payment System). CMS define ‘domains’ for scoring weights which include, inter alia: quality activities, clinical improvement activities, advancing care information performance category, cost/resource, etc. Captured information can be directly used in the CMS sponsored MIPS/MACRA program which may increase payments to the providers.

Process 100 can also be used for providing ongoing feedback system aided by augmented intelligence to each designated entity of a given healthcare eco-system. Process 100 can provide a smart, intuitive, role-based access telehealth interface for all participants.

In one example, a process can collect the information necessary to submit a complete and cohesive claim immediately after the moment of telehealth care. With access to the EHR, the details of the telehealth session, the payor's information, and the provider's information, The process can integrate these sets of information to create the 837 Professional Healthcare Claim and electronically submits the claim. Depending upon the payor's information, the 837 Claim can be sent to either directly to the payor or to a gateway system for submission. Claims submitted in this automated manner can be tracked to manage and identify outstanding and/or unresolved claims.

A home tele-clinic can be provided to aid the physicians in their diagnosis. The tele-clinic can directly provide the physician secure access to real-time consultation, as well as, medical device readings. Accordingly, the physician is armed with adequate information to make a reliable diagnosis and provide required treatment. Physicians can subscribe to the network and provide their tele-clinic availability schedule. Available physicians can be mapped to the patients based on several criteria such as specialization, license in the state, Insurance, ratings etc. A service center can be provided that ensures that the details of the service and complete sessions are archived and made available through open interfaces to those identified by the patient.

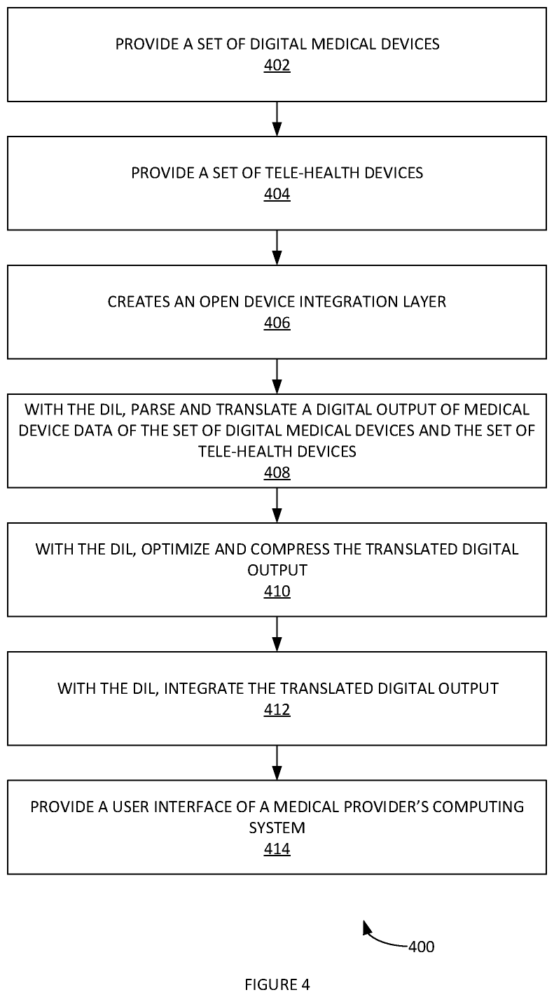

FIG. 4 illustrates an example process for implementing a language neutral virtual assistant, according to some embodiments.

In step 402, process 400 provides a set of digital medical devices, wherein the set of digital medical devices obtains a specified medical sensor information from a patient and communicates the specified medical sensor information to one or more remote entities.

In step 404, process 400 provides a set of tele-health devices, wherein the set of tele-health devices communicates an audio-video data of a tele-health session to the one or more remote entities.

In step 406, process 400 creates an open Device Integration Layer (DIL), wherein the DIL communicates with the set of digital medical devices, and wherein the DIL communicates with the set of tele-health devices in a tele-health session.

In step 408, process 400, with the DIL, parses and translates a digital output of the medical device data of the set of digital medical devices and the set of tele-health devices.

In step 410, process 400 with the DIL, optimizes and compresses the translated digital output of the medical device data of the set of digital medical devices and the set of tele-health devices;

In step 412, process 400, with the DIL, integrates the translated digital output of the set of digital medical devices regardless of any output format of any of the digital medical devices; and

In step 414, process 400 provides a user interface of a medical provider's computing system, wherein the integrated data of the set of digital medical devices is represent by each type of digital medical devices in a canonical form with the audio-video data of the tele-health session.

Canonical format can mean that each device will have a specific format for the data being transmitted—for example, all temperature measurements will be in Centigrade. In an implementation, a higher-level structure can/will be used such as XML or JSON format.

Additionally, process 400 can gather of both objective data/information as well as subjective information.

Example Machine Learning Implementations

Machine learning is a type of artificial intelligence (AI) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity and metric learning, and/or sparse dictionary learning. Random forests (RF) (e.g. random decision forests) are an ensemble learning method for classification, regression and other tasks, that operate by constructing a multitude of decision trees at training time and outputting the class that is the mode of the classes (e.g. classification) or mean prediction (e.g. regression) of the individual trees. RFs can correct for decision trees' habit of overfitting to their training set. Deep learning is a family of machine learning methods based on learning data representations. Learning can be supervised, semi-supervised or unsupervised.

Machine learning can be used to study and construct algorithms that can learn from and make predictions on data. These algorithms can work by making data-driven predictions or decisions, through building a mathematical model from input data. The data used to build the final model usually comes from multiple datasets. In particular, three data sets are commonly used in different stages of the creation of the model. The model is initially fit on a training dataset, that is a set of examples used to fit the parameters (e.g. weights of connections between neurons in artificial neural networks) of the model. The model (e.g. a neural net or a naive Bayes classifier) is trained on the training dataset using a supervised learning method (e.g. gradient descent or stochastic gradient descent). In practice, the training dataset often consist of pairs of an input vector (or scalar) and the corresponding output vector (or scalar), which is commonly denoted as the target (or label). The current model is run with the training dataset and produces a result, which is then compared with the target, for each input vector in the training dataset. Based on the result of the comparison and the specific learning algorithm being used, the parameters of the model are adjusted. The model fitting can include both variable selection and parameter estimation. Successively, the fitted model is used to predict the responses for the observations in a second dataset called the validation dataset. The validation dataset provides an unbiased evaluation of a model fit on the training dataset while tuning the model's hyperparameters (e.g. the number of hidden units in a neural network). Validation datasets can be used for regularization by early stopping: stop training when the error on the validation dataset increases, as this is a sign of overfitting to the training dataset. This procedure is complicated in practice by the fact that the validation dataset's error may fluctuate during training, producing multiple local minima. This complication has led to the creation of many ad-hoc rules for deciding when overfitting has truly begun. Finally, the test dataset is a dataset used to provide an unbiased evaluation of a final model fit on the training dataset. If the data in the test dataset has never been used in training (for example in cross-validation), the test dataset is also called a holdout dataset.

Additional Example Computing Systems

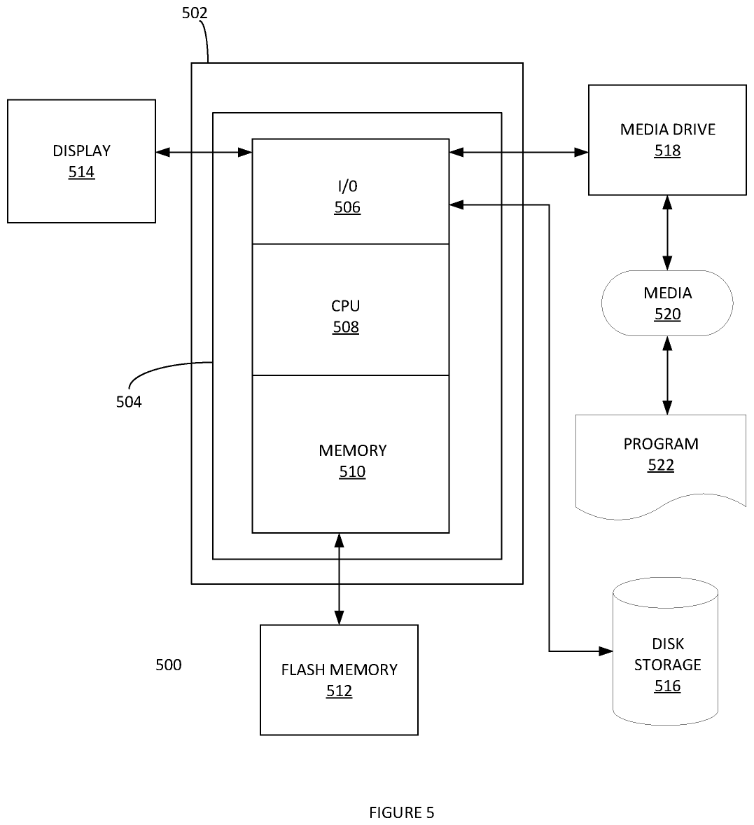

FIG. 5 depicts an exemplary computing system 500 that can be configured to perform any one of the processes provided herein. In this context, computing system 500 may include, for example, a processor, memory, storage, and I/O devices (e.g., monitor, keyboard, disk drive, Internet connection, etc.). However, computing system 500 may include circuitry or other specialized hardware for carrying out some or all aspects of the processes. In some operational settings, computing system 500 may be configured as a system that includes one or more units, each of which is configured to carry out some aspects of the processes either in software, hardware, or some combination thereof.

FIG. 5 depicts computing system 500 with a number of components that may be used to perform any of the processes described herein. The main system 502 includes a motherboard 504 having an I/O section 506, one or more central processing units (CPU) 508, and a memory section 510, which may have a flash memory card 512 related to it. The I/O section 506 can be connected to a display 514, a keyboard and/or other user input (not shown), a disk storage unit 516, and a media drive unit 518. The media drive unit 518 can read/write a computer-readable medium 520, which can contain programs 522 and/or data. Computing system 500 can include a web browser. Moreover, it is noted that computing system 500 can be configured to include additional systems in order to fulfill various functionalities. Computing system 500 can communicate with other computing devices based on various computer communication protocols such a Wi-Fi, Bluetooth® (and/or other standards for exchanging data over short distances includes those using short-wavelength radio transmissions), USB, Ethernet, cellular, an ultrasonic local area communication protocol (e.g. near field communications (NFC), etc.

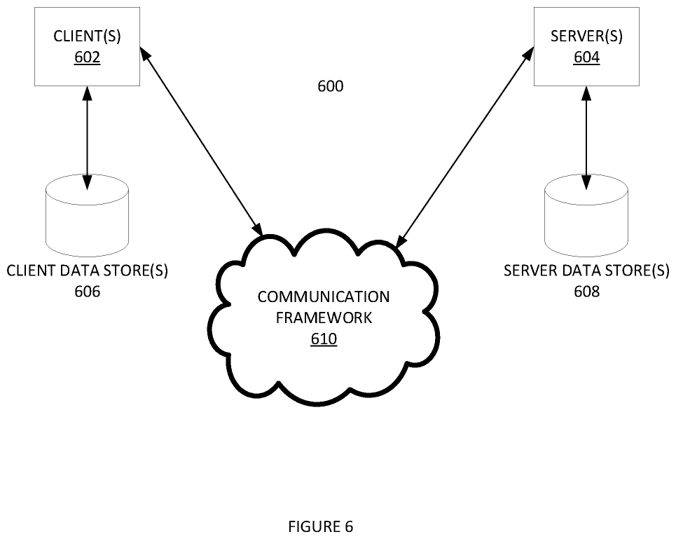

FIG. 6 is a block diagram of a sample computing environment 600 that can be utilized to implement various embodiments. The system 600 further illustrates a system that includes one or more client(s) 602. The client(s) 602 can be hardware and/or software (e.g., threads, processes, computing devices). The system 600 also includes one or more server(s) 604. The server(s) 604 can also be hardware and/or software (e.g., threads, processes, computing devices). One possible communication between a client 602 and a server 604 may be in the form of a data packet adapted to be transmitted between two or more computer processes. The system 600 includes an orchestration framework 610 that can be employed to facilitate communications between the client(s) 602 and the server(s) 604. The client(s) 602 are connected to one or more client data store(s) 606 that can be employed to store information local to the client(s) 602. Similarly, the server(s) 604 are connected to one or more server data store(s) 608 that can be employed to store information local to the server(s) 604. In some embodiments, system 600 can instead be a collection of remote computing services constituting a cloud-computing platform.

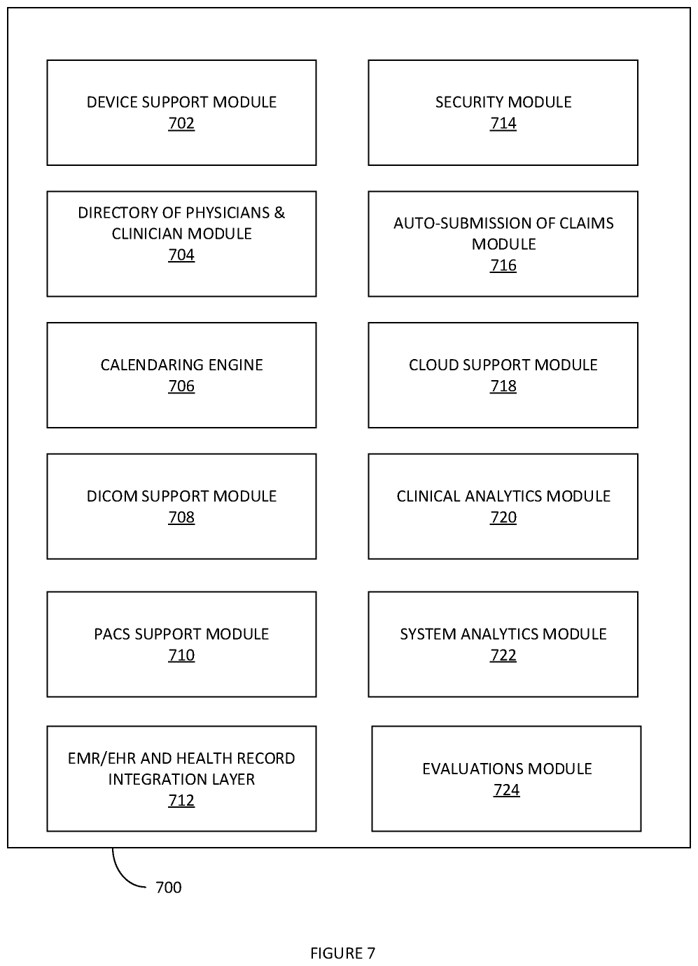

FIG. 7 illustrates an example an integrated telehealth platform system 700, according to some embodiments. Device support module 702 can integrate multiple third-party medical devices into its system based on ecosystem needs through an open device integration layer. Device support module 702 can create a standardized workflow that can guide the operator through discrete steps to perform a given action, or to be able to compare steps already taken with the steps that should be taken. For example, the American Medical Association (AMA) publishes a set of guidelines to be taken for a given episode of care.

Directory of Physicians and clinicians 704 can include providers available to call/involve in a given episode of care, the system maintains a directory of providers. This information can also be integrated through open interfaces with third party partners.

Calendaring engine 706 can schedule calendars and honor relevant time constraints. Calendar owners can include, inter alia: providers, patients, clinicians, facility, and teleclinic resources.

DICOM Support module 708 can DICOM support. As part of the device support, the platform can enable user to view, create, annotate, manipulate, etc. DICOM formatted images. DICOM Support module 708 can enable extensions to DICOM standards for real-time telehealth data transmissions.

PACS Support module 710 can enable picture archiving and communication systems (PACS). PACS is a medical imaging storage technology that provides economical storage and convenient access to images from multiple source machine types (e.g. multiple modalities). PACS Support module 710 can connect with existing PACS systems and/or provide its own PACS system.

EHR and Health Record Integration layer 712 can be used to manage EHRs. EHRs are a digital version of the paper charts in a clinician's office. An EHR 306 can include the medical treatment and history of a patient. An Electronic Health Records (EHR) extends an EHR 306 has a broader view of a patient's care viewable by all participating physicians as well as the patient himself. Information can include items such as allergies, lab results, clinicians' notes, etc. System 1300 can interoperate with other third party EHR, as well as, other commonly used applications in the field through an open workflow integration interface.

Security module 714 can provide a system to handle authentication and authorization, as well as Multi-Factor Authentication (MFA). Additionally, security module 714 can be used for identification of user roles (such as Physician, Clinician, patient/resident, HIPAA-authorized member, non-authorized member, system admin, etc.). Security module 714 monitor and ensure HIPAA compliance.

Auto-submission of claims module 716 can integrate with the payor's electronic claim submission (e.g. patient payor facility 308 via submit to gateway/payor 312). Auto-submission of claims module 716 can automatically determine the CPT/HCPCS based upon the activities of a given session between a physician and a patient and submit the claim on behalf of the patient. Auto-submission of claims module 716 can also track those submitted claims with the reimbursement status to determine outstanding, unresolved claims.

Cloud Support module 718 can maintain and manage information in a cloud-computing environment and enable the information to be accessed via services.

Clinical analytics module 720 can provide various levels of analytics including basic statistics for an individual as well as population-based statistics.

System analytics module 722 can ensure smooth, continuous operation at diagnostic levels, the system can monitor itself and perform self-adjustments, based on data-supported policies.

Evaluations module 724 can support ACO (Accountable Care Organization) needs and requirements for shared savings. Evaluations module 724 can collect and monitor aspects of a healthcare organization and produce reports in support of ACOs. Evaluations module 724 can provide an infrastructure that allows all participants to rate/evaluate the system and the other participants directly involved with a healthcare session.

The implementation of the different modules of system 1300 can be incorporated in three different entities in a telehealth ecosystem. Telehealth agent 202 can be module present at the patient clinic, operated by a clinician or nurse practitioner. Telehealth agent 202 provides telehealth information to the cloud servers and remote entities. telehealth agent 202 procures various device data, multi-media data converts the data into standard, canonical format as defined by a Device Integration Layer Interface 210.

Cloud services can provide secure, cloud storage, archival, real-time media transport and forwarding and portal services for various components.

A distribution engine can manage intelligent distribution of information from the platform bus. Distribution engine can manage distribution such as, inter alia: information access, retrieval, portal views, handling of viewing modalities, etc. These can be handled within cloud servers. SDS-E caters to rendering functionalities for all users of the bus. Role-based access is provided for all data types through managed portal for all participants of the ecosystem.

Various open interfaces can be provided. System 1300 provides open interfaces that enable integration of other third-party devices, protocols, EHRs, applications and services. Device integration layer resides in the Agent and is the interface with which other third-party devices integrate. Multi-media interface incorporates the drivers for the devices responsible for streaming conference audio and video from the camera. Workflow and health records integration layer manages open APIs that map platform data to other EHR systems and workflow applications. The driver to remap to the native system (e.g. EPIC) can be provided by the platform for ease of adoption. Analytics Interface (e.g. Ginger eXchange, Northbound APIs, etc.) can enable exporting platform data to third party and southbound APIs that enable receiving third party analytics and data within the platform.

In one example, system 700 can enable a system consisting of multiple, digitally enabled medical devices such as audio and imaging devices, discrete data devices, and static data devices. The system 700 is able to send digital information to a system 700 portal during an n-way telemedicine session. The information is rendered in real-time in diagnostic quality. The data feeds are managed with differing techniques due to the nature of the information flow. For example, a stream of information supporting devices such as heartbeat or simple scalar numeric information such as blood pressure or temperature. Determining which of the measurements are rendered to the foreground is determined by a few methods, including, inter alia:

1) dynamically, based upon various analytics specific to the patient;

2) past history of the patient;

3) similar case history patients and/or geographical patient data;

4) directed manually by the physician or the technician;

5) available bandwidth and device which is used by the physician to display; and/or

6) explicit instructions from the physician.

When determining which measurement is rendered to the foreground, analytics can influence which measurement is brought forward. For example, in accordance with the alert thresholds that are established for the individual patient, if a threshold is violated (exceeds the boundary), this measurement is brought forward with the appropriate warning regarding the threshold violation. If no threshold violation is detected, inferences about a specific patient will determine which measurement will be brought to the foreground. Based on patient history, age, comorbidities, etc., the most relevant sets of measurements will be presented. Lastly, a physician can manually request to view the measurements s/he wishes to see and can establish that view as the default for a given patient. The rendering of diagnostic level data on the portal is managed concurrently with an active telemedicine session in which the patient (along with a practitioner) and a physician are participating in an audio and video streaming session. Additional participants can be requested to join the telemedicine session creating an n-way telemedicine session. Additional participants may include another physician, a specialist, a family member, and/or friend or guardian.

### CONCLUSION

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g., embodied in a machine-readable medium).

In addition, it can be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

1. A computerized method useful for implementing a language neutral virtual assistant comprising:
providing a set of digital medical devices, wherein the set of digital medical devices obtains a specified medical sensor information from a patient and communicates the specified medical sensor information to one or more remote entities;
providing a set of tele-health devices, wherein the set of tele-health devices communicates an audio-video data of a tele-health session to the one or more remote entities;
creating an open Device Integration Layer (DIL), wherein the DIL communicates with the set of digital medical devices, and wherein the DIL communicates with the set of tele-health devices in a tele-health session;
with the DIL, parsing and translating a digital output of the medical device data of the set of digital medical devices and the set of tele-health devices;
with the DIL, optimizing and compressing the translated digital output of the medical device data of the set of digital medical devices and the set of tele-health devices;
with the DIL, integrating the translated digital output of the set of digital medical devices regardless of any output format of any of the digital medical devices; and
providing a user interface of a medical provider's computing system, wherein the integrated data of the set of digital medical devices is represent by each type of digital medical devices in a canonical form with the audio-video data of the tele-health session.

2. The computerized method of claim 1 further comprising:
transporting the integrated data to a vendor-neutral cloud-computing based data storage platform.

3. The computerized method of claim 2 further comprising:
archiving the integrated data in a Health Insurance Portability and Accountability Act of 1996 (HIPAA)-compliant manner.

4. The computerized method of claim 3, wherein the audio/video channel for consultation with the medical provider further comprises additional features comprising a file transfer protocol, a screen sharing protocol and a recording with permissions protocol.

5. The computerized method of claim 4 further comprising:
providing a transportation engine, wherein the transportation engine transmits the integrated data to one or more cloud-computing platform-based servers for distribution a set of specified remote entities, wherein the set of specified remote entities

6. The computerized method of claim 5 further comprising:
with the DIL, providing a device agnostic, plug-n-play platform-bus that supports an enumerated set of healthcare applications.

7. The computerized method of claim 6, wherein the set of digital medical devices comprises a set of basic-vitals devices.

8. The computerized method of claim 7, wherein the set of digital medical devices comprises a set of waveform devices.

9. The computerized method of claim 8, wherein the set of digital medical devices comprises a set of imaging devices.

10. The computerized method of claim 9, wherein the set of digital medical devices comprises a set of streaming media devices.

11. The computerized method of claim 1 further comprising:
enabling a set of multiple communication channels for transmitting a device telemetry data of the set of digital medical devices and audio-video data to a medical provider computing device for real-time consultation and diagnosis.

12. A computer system useful for implementing a language neutral virtual assistant comprising:
a processor;
a memory containing instructions when executed on the processor, causes the processor to perform operations that:
provide a set of digital medical devices, wherein the set of digital medical devices obtains a specified medical sensor information from a patient and communicates the specified medical sensor information to one or more remote entities;
provide a set of tele-health devices, wherein the set of tele-health devices communicates an audio-video data of a tele-health session to the one or more remote entities;
create an open Device Integration Layer (DIL), wherein the DIL communicates with the set of digital medical devices, and wherein the DIL communicates with the set of tele-health devices in a tele-health session;
with the DIL, parse and translate a digital output of the medical device data of the set of digital medical devices and the set of tele-health devices;
with the DIL, optimize and compress the translated digital output of the medical device data of the set of digital medical devices and the set of tele-health devices;
with the DIL, integrate the translated digital output of the set of digital medical devices regardless of any output format of any of the digital medical devices; and
provide a user interface of a medical provider's computing system, wherein the integrated data of the set of digital medical devices is represent by each type of digital medical devices in a canonical form with the audio-video data of the tele-health session.

13. The computerized system of claim 12, wherein the memory containing instructions when executed on the processor, causes the processor to perform operations that:
transporter the integrated data to a vendor-neutral cloud-computing based data storage platform; and
archives the integrated data in a Health Insurance Portability and Accountability Act of 1996 (HIPAA)-compliant manner.

14. The computerized system of claim 13, wherein the audio/video channel for consultation with the medical provider further comprises additional features comprising a file transfer protocol, a screen sharing protocol and a recording with permissions protocol.

15. The computerized system of claim 14 further comprising:
providing a transportation engine, wherein the transportation engine transmits the integrated data to one or more cloud-computing platform-based servers for distribution a set of specified remote entities, wherein the set of specified remote entities

16. The computerized system of claim 15, wherein the memory containing instructions when executed on the processor, causes the processor to perform operations that:
with the DIL, provides a device agnostic, plug-n-play platform-bus that supports an enumerated set of healthcare applications.

17. The computerized system of claim 16, wherein the set of digital medical devices comprises a set of basic-vitals devices.

18. The computerized system of claim 17, wherein the set of digital medical devices comprises a set of waveform devices.

19. The computerized system of claim 18, wherein the set of digital medical devices comprises a set of imaging devices.

20. The computerized system of claim 12, wherein the memory containing instructions when executed on the processor, causes the processor to perform operations that:
enables a set of multiple communication channels for transmitting a device telemetry data of the set of digital medical devices and audio-video data to a medical provider computing device for real-time consultation and diagnosis.
